(19) United States
(12) Patent Application Publication
(10) Pub. No.: US 2026/0041130 A1
(43) Pub. Date: Feb. 12, 2026

(54) LOIGOLACTOBACILLUS CORYNIFORMIS SUBSP. CORYNIFORMIS AND APPLICATION THEREOF IN IMPROVEMENT ON QUALITY OF PICKLES

(71) Applicant: Zhejiang University of Technology, Hangzhou (CN)

(72) Inventors: Yan Wang, Hangzhou (CN); Xifan Yu, Hangzhou (CN); Peilong Sun, Hangzhou (CN); Hao Yan, Hangzhou (CN)

(73) Assignee: Zhejiang University of Technology, Hangzhou (CN)

(21) Appl. No.: 19/310,874

(22) Filed: Aug. 26, 2025

(30) Foreign Application Priority Data
Aug. 29, 2024 (CN) 202411196226.3

(51) Int. Cl. A23L 19/20; A23L 5/20; C12N 1/20; C12R 1/01

(52) U.S. Cl. CPC A23L 19/20; A23L 5/20; C12N 1/205; A23V 2400/129; C12R 2001/01

(57) ABSTRACT

Disclosed in the present disclosure are Loigolactobacillus coryniformis subsp. coryniformis and an application thereof to improve the quality of pickles. The strain has a good fermenting property, has relatively good acid resistance and salt resistance, can degrade nitrite, and improves the food safety of the pickles. The strain is inoculated to ferment the pickles, which can greatly improve the success rate of fermentation, improve the flavor of the pickles to a certain extent, and effectively improve the quality of the pickles.

### TECHNICAL FIELD

The present disclosure relates to the technical field of microorganisms, and in particular to Loigolactobacillus coryniformis subsp. coryniformis having a stronger fermenting property and an application thereof in improvement on quality of pickles.

### BACKGROUND

Pickles are a kind of typical traditional Chinese fermented food, which are usually fermented by lactic acid bacteria, yeast and other microorganisms by taking fresh vegetables as raw materials, such as Chinese cabbages, mustard, and radishes. It has been proved that original nutrients of vegetables can be retained to a great extent after the vegetables are subjected to microbial fermentation, and the vegetables can also be endowed with a unique fermentation flavor. In addition, the pickles are reported to have the effects of being beneficial to an intestinal environment, promoting human growth and metabolism, relieving intestinal inflammation, and lowering cholesterol in a human body. Mustards after microbial fermentation will produce a special fresh flavor, which not only can promote gastrointestinal digestion, increase appetite, but also has a special spicy taste when eaten because containing isothiocyanate, is crispy and refreshing, and has relatively high edible value.

In addition to several common pickles, there is also a kind of smelly pickles with a special flavor in Ningbo, Taizhou and other places in Zhejiang Province, which is called “Qianli Piaoxiang” pickles. Because of the unique taste and flavor, the pickles are also loved by local consumers. Due to lack of research on this kind of pickles, the corresponding fermentation mechanism is not clear, and the specific fermentation flora is also uncertain. The fermentation success rate of the pickles under natural fermentation for 2-3 months is only 20%, and it is difficult to realize large-scale production with the low fermentation success rate. Furthermore, in the process of pickle processing, because of the long fermentation time, various microbial species, complex succession of colony structures, and diverse metabolic pathways, the quality and safety of the pickles are difficult to unify, resulting in unstable product quality. By performing innoculation and fermentation by artificially adding purified and optimized lactic acid bacteria strains, such that dominant strains can be quickly formed through fermentation, the growth of spoilage bacteria is inhibited, and the defects of natural fermentation are overcome, thus obtaining products with relatively stable quality.

Microorganisms are key factors in fermentation of the pickles, and bacteria are the key microorganisms. According to previous studies, the main bacterial phyla found in the pickles are Firmicutes and Proteobacteria, and Lactobacillus, Weissella, Leuconostoc, etc. are the main genera. Among these dominant genera, the Lactobacillus has been identified as the dominant microflora, which dominates the fermentation process of the pickles. At present, the microflora structure and the fermentation strain of the “Qianli Piaoxiang” pickles are still unclear. Fermentation of the pickles is a high-salt and high-acid environment, exotic bacteria may not have good fermentation characteristics, among which the original lactic acid bacteria in the pickles have stronger environmental adaptability after long-term natural screening, more suitable for inoculation fermentation of the pickles.

Therefore, the lactic acid bacteria with excellent fermenting properties are screened from the successfully fermented “Qianli Piaoxiang” pickles as sample sources, which has important practical significance for improving the flavor of the pickles and solving the problem of quality fluctuation of pickles caused by spice differences.

### SUMMARY

An objective of the present disclosure is to provide Loigolactobacillus coryniformis subsp. coryniformis 523L5 and an application thereof in improvement on quality of fermented pickles. The strain has a good fermenting property, has relatively good acid resistance and salt resistance, can degrade nitrite, and improves the food safety of the pickles. The strain is inoculated to ferment the pickles, which can greatly improve the success rate of fermentation, improve the flavor of the pickles to a certain extent, and effectively improve the quality of the pickles.

The technical solutions employed by the present disclosure are as follows:

The present disclosure provides Loigolactobacillus coryniformis subsp. coryniformis 523L5, being preserved in China General Microbiological Culture Collection Center on Jun. 24, 2024, with the preservation number of CGMCC No. 31070, and the address of No. 3, Yard 1, Beichen West Road, Chaoyang District, Beijing City.

The colony of the Loigolactobacillus coryniformis subsp. coryniformis 523L5 of the present disclosure is round, translucent, white, moist and smooth, and has regular edges when observed by naked eyes. Under an optical microscope, Loigolactobacillus coryniformis is blue-purple, and belongs to Gram-positive bacteria, rod-shaped.

The present disclosure further provides an application of the Loigolactobacillus coryniformis subsp. coryniformis 523L5 in improvement on quality of fermented pickles. Raw materials of the fermented pickles include mustard stems and Chinese cabbages.

Further, a method of the application includes: washing and draining fresh raw materials of fermented pickles, cutting the raw materials into pieces of appropriate sizes, placing the pieces into a pickle jar, adding brine with a mass concentration of 2-4%, performing mixing, inoculating the Loigolactobacillus coryniformis subsp. coryniformis 523L5, performing sealing, and then, performing fermentation at 25-35° C. for 3 months.

Further, the volume of the brine added is 1-2 mL/g based on the mass of the drained pickle raw materials, preferably 1.2 mL/g, and a mass concentration of the brine is preferably 3%.

Further, the Loigolactobacillus coryniformis subsp. coryniformis 523L5 is inoculated in the form of a sterile physiological saline bacterial suspension of wet bacterial cells, the wet bacterial cells has a concentration of 0.5-5 g/L (preferably 1 g/L), and an inoculation amount is 1-3 mL/100 g based on the weight of the drained pickle raw materials.

Further, the wet bacterial cells are prepared according to the following method: inoculating the Loigolactobacillus coryniformis subsp. coryniformis 523L5 into a MRS solid medium plate, performing activation and culture at 37° C. for 48 h, then, inoculating a cultured bacterial solution into a MRS liquid medium for culture at 37° C. for 16 h, inoculating the cultured bacterial solution into a MRS liquid medium at an inoculation amount of 2% by volume concentration, performing culture in a shaker at 37° C. until OD_{600 nm} is 1.0, centrifuging the culture solution at 4° C. and 5000 rpm for 10 min, removing supernatant, washing bacterial cells repeatedly with sterile physiological saline three times, collecting wet bacterial cells, and suspending the wet bacterial cells in sterile physiological saline of the same volume to be prepared into a bacterial suspension as a mother starter for fermentation of the pickles.

Compared with the prior art, the beneficial effects of the present disclosure are mainly embodied in:
(1) The strain of the present disclosure can shorten a fermentation period of the pickles, the Loigolactobacillus coryniformis subsp. coryniformis 523L5 is screened from a “Qianli Piaoxiang” pickle solution, which has good growth performance, relatively high acid production capacity, relatively strong salt tolerance and acid tolerance, and can tolerate salinity of about 7%. The strain can grow in the MRS liquid medium with pH of 4.5 and has a good fermenting property. Inoculation of the Loigolactobacillus coryniformis subsp. coryniformis 523L5 makes the success rate of fermented pickles significantly higher than that of naturally fermented pickles, which can significantly improve the pH value of the fermentation broth of the pickles, reduce the acidity by 42.86%, achieve higher maturity in a shorter fermentation period, and shorten the fermentation period of the pickles while the sensory quality is ensured.
(2) The strain of the present disclosure can improve the fresh flavor of the fermented pickles. Fresh flavor amino acids such as aspartic acid and glutamic acid in the pickles are improved, and most bitter amino acids are reduced, such that the fresh flavor of the pickles is improved to a certain extent.
(3) The strain of the present disclosure can reduce the pungent sourness of the pickles, increase the content of organic acids such as acetic acid and succinic acid of the fermented pickles, endow the pickles with softer sourness, and reduce the pungent sourness of the pickles to a certain extent.
(4) The strain of the present disclosure can improve the flavor of the fermented pickles, 51 kinds of volatile flavor substances in the fermented pickles are fermented through inoculation of the Loigolactobacillus coryniformis subsp. coryniformis, which increases 15 kinds of volatile flavor substances compared with volatile flavor substances of naturally fermented pickles. Moreover, the contents of acids and sulfur compounds are obviously increased, where the contents of butanol and n-hexanol occupy the main position in alcohols, and butyric acid replaces n-hexanoic acid to become the acid substance with the most content. The bad smell of fat of the pickles can be reduced. The increase of the dimethyl disulfide content can also increase the onion flavor and the vegetable flavor of the pickles, indicating that the Loigolactobacillus coryniformis subsp. coryniformis can improve the flavor and the quality of the pickles.

To sum up, the pickles fermented through the Loigolactobacillus coryniformis subsp. coryniformis 523L5 of the present disclosure have a higher pH value, lower acidity, and higher maturity in a shorter fermentation period, sensory evaluation of the pickles fermented through the Loigolactobacillus coryniformis subsp. coryniformis 523L5 is not inferior to that of the pickles fermented naturally in terms of quality and flavor, the pickles have the higher umami amino acid content, the lower bitter amino acid content, and the higher content of acetic acid and succinic acid with softer sour taste. Compared with the naturally fermented pickles, the flavor of the pickles fermented through the Loigolactobacillus coryniformis subsp. coryniformis 523L5 has no significant difference, and the types and relative contents of volatile flavor substances (especially alcohols and acids) in the pickles are improved, such that the quality of the pickles can be effectively improved, and the strain has good application potential in the fermentation of the pickles.

### BRIEF DESCRIPTION OF THE DRAWINGS

FIG. 1 shows a graph of colony morphology (A) and a graph of cell morphology (B) of a strain 523L5.

FIG. 2 shows a phylogenetic tree of a strain 523L5.

FIG. 3 shows growth characteristics of a strain 523L5, where (a) shows a growth curve, and (b) shows an acid production rate curve.

FIG. 4 shows a salt tolerance curve of a strain 523L5.

FIG. 5 shows an acid tolerance curve of a strain 523L5.

### DETAILED DESCRIPTIONS OF THE EMBODIMENTS

The present disclosure is further described below in conjunction with particular examples, but the protection scope of the present disclosure is not limited herein:

MRS liquid medium: 10.0 g of peptone, 8.0 g of beef extract powder, 5.0 g of yeast extract powder, 20.0 g of glucose, 2.0 g of dipotassium hydrogen phosphate, 2.0 g of diammonium hydrogen citrate, 5.0 g of sodium acetate, 0.2 g of magnesium sulfate, 0.04 g of manganese sulfate, and 1.0 mL of Tween-80 are dissolved in 1000 mL of distilled water under pH of 5.7±0.2 and sterilized at 118° C. for 20 min.

MRS solid medium: 10.0 g of peptone, 8.0 g of beef extract powder, 5.0 g of yeast extract powder, 20.0 g of glucose, 2.0 g of dipotassium hydrogen phosphate, 2.0 g of diammonium hydrogen citrate, 5.0 g of sodium acetate, 0.2 g of magnesium sulfate, 0.04 g of manganese sulfate, 1.0 mL of Tween-80, and 14.0 g of agar are dissolved in 1000 mL of distilled water under pH of 6.5±0.2 and sterilized at 121° C. for 20 min.

Nitrite degradation test medium: 2 mL of 5 mg/mL NaNO₂ aqueous solution is added to 50 mL of MRS liquid medium.

### Example 1: Making of “Qianli Piaoxiang” Pickles

Fresh mustard stems were washed in running tap water, drained, cut into pieces of appropriate sizes, boiled and blanched. After cooling, 1.2 kg of mustard stems were weighed and put into a 3 L pickle jar, and 1% of peppers and 1% of garlic by mass fraction (by weight of mustard stems) were added. 3% of brine solution was added to cover the mustard stems. Sealing was performed, and fermentation was performed at a room temperature (25-30° C.) for 3 months. After completion of the fermentation, the color, texture, flavor and taste of the fermented pickles were scored by trained panelists according to Table 1, and the pickles with a total score of more than 70 were defined as excellent fermented pickles and used as samples for subsequent isolation of strains.

TABLE 1
Scoring standards

| Index | Scoring standard | Score |
|---|---|---|
| Color | Fermentation broth being turbid, poor in color, and | 21-25 |
| | having a floating film on surface | |
| | Fermentation broth being slight turbid and dark in color | 11-20 |
| | Fermentation broth being clear and bright in color | 0-10 |
| Texture | Texture being soft and tender with almost no brittleness | 21-25 |
| | Texture being relatively soft with poor brittleness | 11-20 |
| | Texture being relatively hard with good brittleness | 0-10 |
| Flavor | No obvious sour taste, and being smelly and rich in | 21-25 |
| | special flavor | |
| | Smelly with a hint of sourness and a slight special flavor | 11-20 |
| | of this kind of pickles | |
| | Not smelly, only having a sour flavor, or having | 0-10 |
| | unpleasant odor without special flavor of this kind of | |
| | pickles | |
| Taste | Having typical flavor of this kind of pickles with | 21-25 |
| | relatively good taste | |
| | Having typical flavor of this kind of pickles, but being | 11-20 |
| | overly sour or sour with poor taste | |
| | Having no typical flavor of this kind of pickles, but only | 0-10 |
| | having sourness or unpleasant odor | |

### Example 2: Screening and Identification of Loigolactobacillus coryniformis Subsp. Coryniformis 523L5

1. Primary screening:

“Qianli Piaoxiang” pickles solution screened in Example 1 was diluted in a 10-fold gradient with sterile physiological saline, 100 μL of each of the 100 (i.e. stock solution), 10-3 and 10-4 dilution samples were pipetted, and evenly coated on a MRS solid medium plate with a sterile coating rod. After culture at 37° C. for 48 h, colonies with calcium dissolving rings and different morphology were selected and repeatedly streaked on the MRS solid medium plate, and finally, single colonies were screened and marked as strains 523L1, 523L5 and 523L12. The purified strains were cryopreserved in 20% glycerol.

### 2. Secondary Screening:

A ring of bacterial solution cryopreserved in a glycerol tube in step 1 was taken with a sterile inoculation loop and streaked on the MRS solid medium plate. After culture at 37° C. for 48 h, a ring of single colonies was taken and mixed evenly in a test tube containing a MRS liquid medium. Culture was performed at 37° C. for 24 h. The bacterial density (OD_{600 nm}) and pH value of samples were determined at 12 h and 24 h respectively. See Table 2 for results.

2 mL of activation culture solution (OD_{600 nm}=1.0) was taken and put into 50 mL of sterilized nitrite degradation test medium. After culture at 37° C. for 70 h, the nitrite content was determined by a naphthalene ethylenediamine hydrochloride method, and the nitrite degradation rate of each strain was calculated. See Table 2 for the results.

The activated strains were coated in the MRS solid media containing NaCl and 2% calcium carbonate at different mass concentrations (3%, 4%, 5%) respectively, and cultured at 37° C. for 48 h. The appearance of calcium dissolving rings around the colonies, indicating that the strain has salt tolerance. See Table 2 for the concentration results of NaCl tolerance.

TABLE 2
Determination results of fermenting properties of various strains

| | OD_{600 nm} | | pH | | Nitrite degradation | Salt |
|---|---|---|---|---|---|---|
| Strain | 12 h | 24 h | 12 h | 24 h | capacity (%) | tolerance |
| 523L1 | 1.35 | 1.96 | 4.57 | 3.83 | 40.58 | <3% |
| 523L5 | 1.67 | 2.06 | 4.33 | 3.91 | 43.36 | >5% |
| 523L12 | 1.63 | 2.03 | 4.14 | 3.75 | 41.96 | 3% |

The strain 523L5 has good growth capacity, strong acid production capacity, salt tolerance capacity and strong nitrite degradation capacity.

### 3. Identification of Strain 523L5

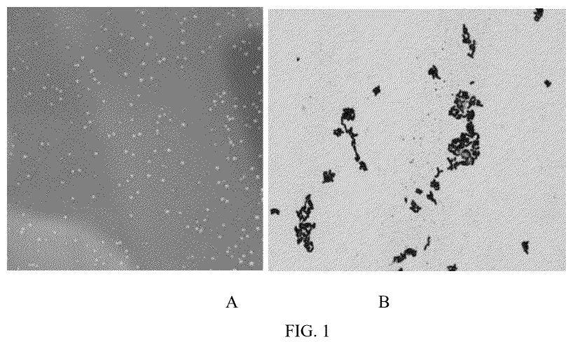

Morphological identification: 100 μL of bacterial solution cryopreserved in the glycerol tube was pipetted and coated on a MRS solid medium plate. After culture at 37° C. for 48 h, the colony morphology was observed with naked eyes, and the cell morphology was observed under an optical microscope after Gram staining. The colony morphology and cell morphology of the strain 523L5 are shown in FIG. 1. The colony is round, translucent, white, moist and smooth with neat edges by visual observation. Under the optical microscope, the strain is bluish purple, and belongs to Gram-positive bacteria, rod-shaped.

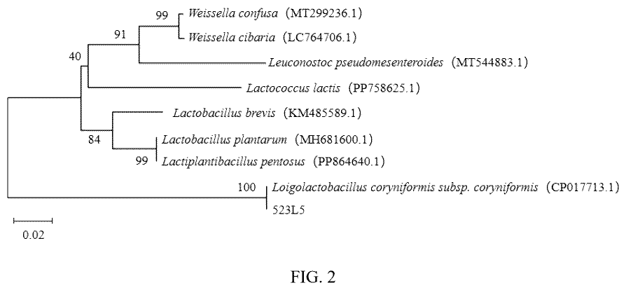

Molecular identification: The strain obtained by screening was inoculated into a test tube containing the MRS liquid medium and cultured in a shaker at 37° C. for 16 h, and bacterial cells were harvested. The DNA was extracted according to a bacterial genome DNA extraction kit, and the DNA was used as a template to amplify the full-length sequence of 16S rDNA with bacterial universal primers (27F: 5′-AGAGTTTGATCCTGGCTCAG-3′ and 1492R: 5′-TACGGCTACCTTGTTACGACTT-3′). PCR products were sent to Beijing Tsingke Biotech Co., Ltd. for sequencing after passing the inspection, BLAST was used for homology comparison, and MEGA10.0 was used for constructing the phylogenetic tree, as shown in FIG. 2.

The 16S rDNA sequence (SEQ ID NO. 1) was introduced into NCBI for BLAST homology alignment, and results showed that the strain 523L5 had 100% similarity with Loigolactobacillus coryniformis subsp. coryniformis. In combination with morphological features, the strain 523L5 was identified as Loigolactobacillus coryniformis subsp. coryniformis 523L5, being preserved in China General Microbiological Culture Collection Center on Jun. 24, 2024, with the preservation number of CGMCC No. 31070, and the address of No. 3, Yard 1, Beichen West Road, Chaoyang District, Beijing City.

### Example 3: Fermenting Property Test of Strain 523L5

### 1. Growth Curve Determination

Growth capacity is an important parameter for evaluating fermentation strains, and a growth curve can intuitively understand the growth rule of bacterial cells.

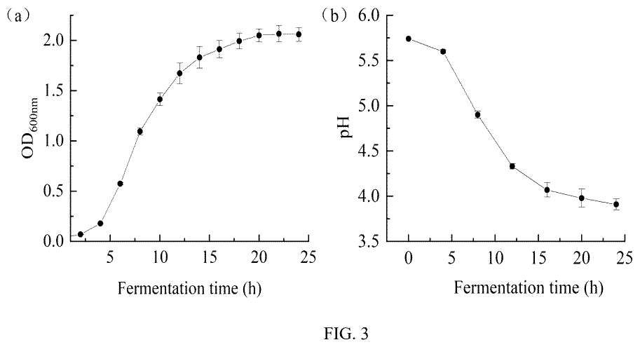

The strain 523L5 was inoculated on a MRS solid medium plate, activated and cultured at 37° C. for 48 h, inoculated in a MRS liquid medium, cultured at 37° C. for 16 h, then, inoculated into a MRS liquid medium at an inoculation amount of 2% by volume concentration, and cultured in a shaker at 37° C. for 24 h. Sampling was performed once every 2 h during culture, a bacterial density (OD_{600 nm}) of the bacterial solution was determined. See FIG. 3a for results. It can be seen that the strain 523L5 is in a growth lag phase and grows slowly at 0-4 h; After 6 h, the strain enters a logarithmic growth phase, and the number of bacterial cells is increased rapidly. After 14 h, the strain grows slowly and begins to enter a stationary phase. Therefore, the Loigolactobacillus coryniformis subsp. coryniformis 523L5 has good growth capacity.

### 2. Determination of Acid Production Rate

In the step 1, during shaking culture at 37° C. for 24 h, sampling was performed once every 4 h to determine a pH value of the bacterial solution. Results are shown in FIG. 3b. It can be seen that the strain 523L5 is in a growth adaptation phase at 0-4 h, and pH is decreased slowly. After the strain 523L5 adapts to a culture environment at 4-16 h, it grows rapidly and is in the logarithmic growth phase, and pH is decreases rapidly. After 16 h, bacteria grow slowly and enter the stationary phase, and pH is decreased slowly and tends to be stabilized.

### 3. Salt Tolerance

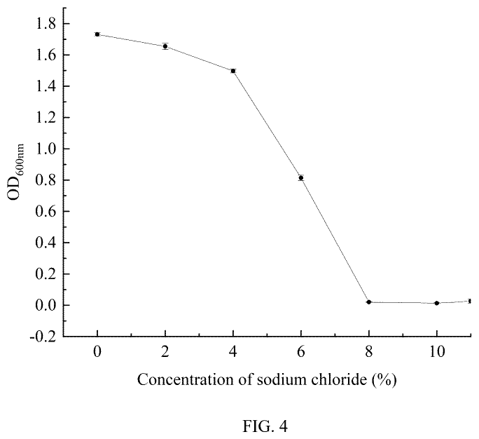

The strain 523L5 was inoculated on a MRS solid medium plate, activated and cultured at 37° C. for 48 h, then, inoculated in a MRS liquid medium and cultured at 37° C. for 16 h, then, inoculated into MRS liquid media supplemented with 0%, 2%, 4%, 6%, 8%, 10%, 12% and 14% of NaCl (i.e. salt concentration) at an inoculation amount of 2% by volume concentration and cultured at 37° C. for 24 h. An OD_{600 nm} value of the bacterial solution was determined. Results are shown in FIG. 4.

As can be seen from FIG. 4, the strain 523L5 is less inhibited when the salt concentration is 2%-4%, OD_{600 nm} is about 0.8 when the salt concentration is increased to 6%, and the strain 523L5 can hardly grow when the salt concentration is increased to over 8%.

The strain 523L5 is determined to have certain salt tolerance capacity and can tolerate about 7% salt concentration.

### 4. Acid Tolerance

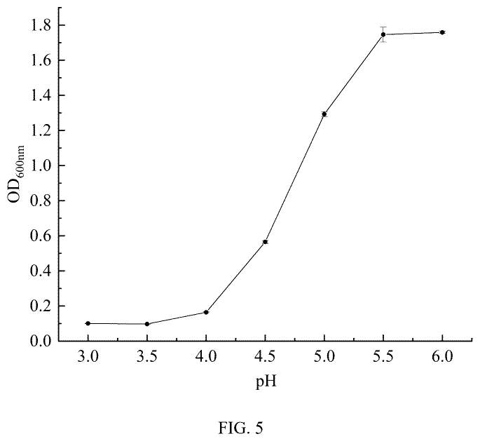

The strain 523L5 was inoculated in a MRS solid medium plate, activated and cultured at 37° C. for 48 h, inoculated in a MRS liquid medium and cultured at 37° C. for 16 h, then, inoculated into MRS liquid media with pH of 3.0, 3.5, 4.0, 4.5, 5.0, 5.5 and 6.0 at an inoculation amount of 2% by volume concentration, and cultured in a shaker at 37° C. for 24 h. The bacterial density (OD_{600 nm}) of each culture solution was determined. Results are shown in FIG. 5.

It can be seen from FIG. 5 that when pH is less than 4.0, the strain 523L5 can hardly grow normally. When pH rises to 4.5, OD_{600 nm} is about 0.6. When pH rises to 5.0, the strain 523L5 is less inhibited, and when pH rises to 5.5 or above, the strain 523L5 can grow normally.

### Example 4: Wet Bacterial Cells of Strain 523L5

The strain 523L5 was inoculated into a MRS solid medium plate, activated and cultured at 37° C. for 48 h, then, inoculated into a MRS liquid medium for culture at 37° C. for 16 h, then, inoculated into a MRS liquid medium at an inoculation amount of 2% by volume concentration, and cultured in a shaker at 37° C. until OD_{600 nm} was 1.0. A culture solution was centrifuged at 4° C. and 5000 rpm for 10 min, supernatant was removed, bacterial cells were washed repeatedly with sterile physiological saline three times, and wet bacterial cells were collected. Finally, the wet bacterial cells were suspended in sterile physiological saline of the same volume and prepared into a bacterial suspension as a mother starter for fermentation of pickles.

### Example 5: Application of Loigolactobacillus coryniformis Subsp. Coryniformis 523L5 in Fermentation of Mustard Stems

1. Making of pickles: Fresh mustard stems were washed, drained, cut into pieces of appropriate sizes, and placed in a pickle jar. Brine with mass concentration of 3% was added according to the amount of 1 g: 1.2 mL and mixed. The strain 523L5 wet bacterial suspension prepared by the method of Example 4 (wet bacterial cell concentration 1 g/L) was inoculated according to the weight of mustard stems to the volume of the bacterial suspension of 100 g: 2 mL as a test group, and naturally fermented stems without inoculation were used as a blank control group. After sealing, fermentation was carried out at 25-35° C. for 3 months.

2. Determination of pH of pickles: The fermentation broth of finished pickles was taken and directly determined by using a pH meter. The pH value of the pickles inoculated with the Loigolactobacillus coryniformis subsp. coryniformis 523L5 after completion of fermentation was 5.57, which was higher than that of the pickles fermented naturally (pH=4.78). Generally speaking, the pickles are considered mature when the pH value of the fermentation broth is less than 4.0, but a small amount of pickles may result in a pH value greater than 4.0 due to different production processes and different fermentation microorganisms. Compared with ordinary pickles, the pH value of “Qianli Piaoxiang” pickles will be higher, and the sour taste will be weak or “Qianli Piaoxiang” pickles has no sour taste.

3. Determination of total acid of pickles: 10 mL of fermentation broth was accurately measured and added to a 100 mL volumetric flask, and water is added to the required scale and mixed well. Filtration was performed with filter paper or absorbent cotton, and filtrate is reserved for later use. 25 mL of filtrate was accurately pipetted into a 100 ml conical flask, a few drops of phenolphthalein indicator was added, titration was performed with a 0.1 mol/L sodium hydroxide aqueous solution until first appearance of pink color, and the endpoint was determined when no fading appeared within 0.5 min. A titration volume of the sodium hydroxide aqueous solution consumed was recorded. The total acid content in the sample was calculated based on lactic acid in grams per liter (g/L).

The total acid content of the pickles inoculated with the Loigolactobacillus coryniformis subsp. coryniformis 523L5 after fermentation is 1.84 g/L, which is decreased by 42.86% compared with the natural fermentation group (3.22 g/L).

4. Determination of organic acid content: 1 mL of fermentation broth of the pickles was transferred to a centrifuge tube, centrifuged at 10000/min for 15 min, and filtered through a 0.22 μm membrane. The organic acid content of filtrate was tested through high performance liquid chromatography (HPLC). See Table 4 for results.

Chromatographic column: Ultimate AQ-C18 (5 μm, 250 mm×4.6 mm), column temperature: 30° C. mobile phase A: 20 mM disodium hydrogen phosphate aqueous solution (adjusted to pH=2.6 with phosphoric acid): methanol volume ratio: 99:1, mobile phase B: methanol, flow rate: 0.7 mL/min, injection volume: 10 μL, and UV detection wavelength: 210 nm. The gradient elution procedure for organic acids is shown in Table 3 below.

TABLE 3
Gradient elution procedure for organic acids

| Time (min) | A (%) | B (%) |
|---|---|---|
| 0 | 100 | 0 |
| 9 | 100 | 0 |
| 20 | 50 | 50 |
| 23 | 20 | 80 |
| 23.1 | 100 | 0 |

TABLE 4
Types and contents of organic acids in pickles

| | Concentration of organic acid in sample (mg/L) | | | | | | | | |
|---|---|---|---|---|---|---|---|---|---|
| | Oxalic | Tartaric | Formic | Malic | Lactic | Acetic | Citric | Succinic | Propanoic |
| Group | acid | acid | acid | acid | acid | acid | acid | acid | acid |
| Control | 8.69 | 7.90 | 580.66 | 56.31 | 87.45 | 1902.77 | 24.72 | 303.13 | 4642.79 |
| group | | | | | | | | | |
| Test | 5.38 | — | 308.26 | 40.79 | 26.38 | 2582.89 | 16.15 | 400.60 | 2798.91 |
| group | | | | | | | | | |

Sour taste is the main flavor of the pickles, which mainly comes from the rich organic acids produced by the fermentation of the pickles. The main organic acids produced from the pickles subjected to fermentation inoculation or the pickles naturally fermented are propanoic acid, acetic acid, formic acid and succinic acid, but the contents are different. The contents of propanoic acid and formic acid in the naturally fermented pickles are higher than those in the pickles subjected to fermentation inoculation, while the contents of acetic acid and succinic acid in the pickles subjected to fermentation inoculation are higher. The propanoic acid has pungent sour taste at a room temperature, while the acetic acid and the succinic acid have mild sour taste, so they can endow the pickles with softer sour taste. The contents of other organic acids in the natural fermented pickles are slightly higher than those in the pickles subjected to inoculation fermentation except for the four kinds of organic acids with higher contents. Therefore, in terms of organic acids, the pickles subjected to inoculation fermentation can achieve sour taste close to that of the naturally fermented pickles.

5. Determination of amino acid content: 1 mL of fermentation broth was centrifuged at 4° C. and 12000 rpm for 10 min, and then filtered with a 0.22 μm filter membrane. Filtrate was analyzed by using an automatic amino acid analyzer to determine the free amino acid content. The free amino acid content is expressed in mg/L, and measurement results are shown in Table 5.

TABLE 5
Types and contents of amino acids in pickles

| | Content (mg/L) | |
|---|---|---|
| | Natural fermentation | Inoculation fermentation |
| Aspartic acid | 7.755 | 11.520 |
| Threonine | 0.243 | 0.487 |
| Serine | — | — |
| Glutamic acid | 7.554 | 10.675 |
| Glycine | 0.528 | 0.153 |
| Aminopropionic acid | 2.440 | — |
| Cysteine | 2.031 | 0.491 |
| Valine | 7.343 | 3.614 |
| Methionine | 1.493 | 0.254 |
| Isoleucine | 1.502 | — |
| Leucine | 3.642 | 0.716 |
| Tyrosine | 3.285 | 2.301 |
| Phenylalanine | 3.265 | 9.147 |
| Lysine | 5.492 | 3.450 |
| Histidine | 1261.725 | 1245.206 |
| Arginine | — | — |
| Proline | 5.613 | 4.112 |

The types and contents of free amino acids in the pickles directly affect the taste of the pickles. Amino acids are divided into four types: being umami (aspartic acid and glutamic acid), sweet (alanine, threonine, glycine and serine), bitter (arginine, histidine, isoleucine, leucine, methionine, phenylalanine, valine, proline and lysine) and tasteless (cysteine and tyrosine). As shown in Table 5, pickles fermented by the Loigolactobacillus coryniformis subsp. coryniformis 523L5 has higher aspartic acid and glutamic acid contents than the naturally fermented pickles, and the aspartic acid and the glutamic acid are the main sources of the umami flavor of the pickles. The Loigolactobacillus coryniformis subsp. coryniformis 523L5 helps to improve the umami flavor of the pickles. For example, the contents of the bitter amino acids such as isoleucine, leucine, methionine and valine in the pickles fermented by the Loigolactobacillus coryniformis subsp. coryniformis 523L5 are reduced, which is conductive to improvement on the overall flavor of the pickles.

6. Sensory evaluation: A sensory evaluation panel composed of well-trained team members from pickle factories score the color, texture, flavor and taste of the fermented pickles of different treatments according to the sensory scoring standards in Table 6. Because of differences of processing technologies and fermentation microorganisms of the pickles, sensory evaluation standards are significantly different from most pickles.

TABLE 6
Sensory scoring standards

| Index | Scoring standard | Score |
|---|---|---|
| Color | Fermentation broth being turbid, poor in color, and | 21-25 |
| | having a floating film on surface | |
| | Fermentation broth being slight turbid and dark in color | 11-20 |
| | Fermentation broth being clear and bright in color | 0-10 |
| Texture | Texture being soft and tender with almost no brittleness | 21-25 |
| | Texture being relatively soft with poor brittleness | 11-20 |
| | Texture being relatively hard with good brittleness | 0-10 |
| Flavor | No obvious sour taste, and being smelly and rich in | 21-25 |
| | special flavor | |
| | Smelly with a hint of sourness and a slight special flavor | 11-20 |
| | of this kind of pickles | |
| | Not smelly, only having a sour flavor, or having | 0-10 |
| | unpleasant odor without special flavor of this kind of | |
| | pickles | |
| Taste | Having typical flavor of this kind of pickles with | 21-25 |
| | relatively good taste | |
| | Having typical flavor of this kind of pickles, but being | 11-20 |
| | overly sour or sour with poor taste | |
| | Having no typical flavor of this kind of pickles, but only | 0-10 |
| | having sourness or unpleasant odor | |

The pickled inoculated with the Loigolactobacillus coryniformis subsp. coryniformis 523L5 have no significant difference in texture and color from the naturally fermented pickles, and scored slightly lower than the naturally fermented pickles in taste and flavor, but there is no significant difference. Results indicate that the Loigolactobacillus coryniformis subsp. coryniformis 523L5 cannot improve the sensory quality of the pickles significantly, but it can guarantee the sensory quality similar to that of the naturally fermented pickles.

7. Determination of volatile flavor substances in pickles: 2 g of pickle solution was taken and added into a 20 mL headspace bottle, 0.72 g of NaCl was added, a 0.6 mg/mL methyl octanoate aqueous solution was used as an internal standard, 100 μL of 0.6 mg/mL methyl octanoate aqueous solution was added into each sample, and volatile flavor substances were tested by a GC-MS system. Firstly, a DVB-PDMS fiber head was adsorbed in the headspace bottle at 40° C. for 30 min, and then inserted into a GC-MS injection port for desorption at 250° C. for 5 min, where a split ratio was 40:1, carrier gas was He, and a flow rate was 1 mL/min.

Volatile substances were separated and tested by using a GC-MS system equipped with a DB-5 MS chromatographic column (30 m×0.25 mm×0.25 μm). The temperature program was as follows: rising to 76° C. (keeping for 1 min) at 4° C./min from 40° C. (keeping for 1 min), rising to 94° C. (keeping for 1 min) at 2° C./min, rising to 114° C. (keeping for 1 min) at 4° C./min, rising to 150° C. (keeping for 1 min) at 5° C./min, and rising to 230° C. (keeping for 5 min) at 10° C./min. The sample injection temperature was 250° C., and the mass spectrum conditions were as follows: full scan acquisition mode, electron impact (EI) ion source, electron energy of 70 eV, ion source temperature of 200° C., quadrupole temperature of 150° C., and mass scanning range of m/z 29-650.

Determination results of volatile flavor substances of the pickles fermented by the Loigolactobacillus coryniformis subsp. coryniformis 523L5 are shown in Table 7.

TABLE 7
Types and relative contents of volatile components in pickles

| Relative content (%) | |
|---|---|
| Inoculation | Natural |
| fermentation | fermentation |

Alcohols

| | | | | |
|---|---|---|---|---|
| 1 | 75-65-0 | Tert butyl alcohol | 0.21 | — |
| 2 | 14898-79-4 | (R)-(−)-2-butanol | 7.68 | 11.30 |
| 3 | 6032-29-7 | 2-pentanol | 0.80 | — |
| 4 | 31087-44-2 | (R)-(−)-2-pentanol | 0.38 | — |
| 5 | 123-51-3 | Isoamyl alcohol | 0.09 | 0.52 |
| 6 | 137-32-6 | 2-methylbutanol | 0.17 | — |
| 7 | 111-27-3 | N-hexanol | 8.65 | 18.14 |
| 8 | 543-49-7 | 2-heptanol | 0.15 | — |
| 9 | 60-12-8 | Phenethyl alcohol | 0.35 | — |
| 10 | 71-36-3 | n-butyl alcohol | — | 1.24 |
| | | Total: | 18.48 | 31.20 |

Ketones

| | | | | |
|---|---|---|---|---|
| 11 | 112-12-9 | 2-undecanone | — | 0.29 |
| 12 | 107-87-9 | 2-pentanone | — | 0.15 |
| 13 | 821-55-6 | 2-nonanone | — | 0.18 |
| 14 | 78-93-3 | 2-butanone | 0.48 | — |
| 15 | 6137-06-0 | 4-methyl-2-heptanone | 0.16 | — |
| 16 | 110-43-0 | 2-heptanone | — | 0.33 |
| | | Total: | 0.64 | 0.96 |

Esters

| | | | | |
|---|---|---|---|---|
| 17 | 623-42-7 | Methyl butyrate | 1.20 | 0.48 |
| 18 | 105-54-4 | Ethyl butyrate | 1.01 | 2.76 |
| 19 | 539-82-2 | Ethyl valerate | — | 0.77 |
| 20 | 624-24-8 | Methyl valerate | 0.21 | 0.17 |
| 21 | 108-64-5 | Ethyl isovalerate | — | 0.30 |
| 22 | 556-24-1 | Methyl isovalerate | — | 0.55 |
| 23 | 644-49-5 | 2-methyl-propanoic acid | 0.41 | 8.20 |
| | | propylester | | |
| 24 | 7452-79-1 | Ethyl 2-methylbutyrate | — | 0.07 |
| 25 | 123-66-0 | Ethyl hexanoate | — | 4.70 |
| 26 | 106-70-7 | Methyl hexanoate | 3.23 | — |
| 27 | 819-97-6 | Butyl butanoate | 0.50 | — |
| 28 | 123-66-0 | Ethyl caproate | 0.80 | — |
| 29 | 106-30-9 | Ethyl oenanthate | — | 0.14 |
| 30 | 106-73-0 | Methyl heptanoate | 0.10 | — |
| 31 | 23361-74-2 | Butyl valerate | 0.08 | — |
| 32 | 626-77-7 | Propyl caproate | 0.58 | — |
| 33 | 626-82-4 | Butyl caproate | 0.69 | — |
| 34 | 626-82-4 | Butyl caproate | 0.52 | — |
| 35 | 57-06-7 | Allyl isothiocyanate | — | 0.33 |
| 36 | 105-66-8 | Propyl butanoate | 0.09 | — |
| 37 | 2639-63-6 | Butyric acid hexylester | 1.62 | 0.19 |
| 38 | 111-11-5 | Methyl octanoate | — | 0.27 |
| 39 | 106-32-1 | Ethyl caprylate | 0.25 | — |
| 40 | 624-13-5 | Propyl caprylate | 0.05 | — |
| 41 | 1117-55-1 | Hexyl caprylate | 0.22 | — |
| 42 | 2432-77-1 | S-methyl thiohexanoate | — | 1.50 |
| | | Total: | 11.56 | 20.43 |

Acids

| | | | | |
|---|---|---|---|---|
| 43 | 79-09-4 | Propanoic acid | 0.09 | — |
| 44 | 79-33-4 | L-lactic acid | 0.14 | — |
| 45 | 107-92-6 | Butanoic acid | 13.86 | 2.63 |
| 46 | 503-74-2 | Isovaleric acid | 0.47 | — |
| 47 | 116-53-0 | 2-methylbutyric acid | 0.64 | — |
| 48 | 109-52-4 | N-pentanoic acid | 11.65 | — |
| 49 | 628-46-6 | 5-methylhexanoic acid | 0.14 | — |
| 50 | 111-14-8 | Heptylic acid | 2.19 | — |
| 51 | 124-07-2 | Caprylic acid | 1.16 | — |
| 52 | 142-62-1 | N-hexanoic acid | — | 15.27 |
| | | Total: | 30.33 | 17.90 |

Sulfur compounds

| | | | | |
|---|---|---|---|---|
| 53 | 10152-76-8 | Allyl methyl sulfide | 0.02 | 1.66 |
| 54 | 624-92-0 | Dimethyl disulfide | 29.08 | 17.19 |
| 55 | 2179-58-0 | Allyl methyl disulphide | 0.05 | — |
| 56 | 3658-80-8 | Dimethyl trisulfide | 3.58 | — |
| 57 | 5756-24-1 | Dimethyltetrasulfane | 0.57 | — |
| 58 | 33577-16-1 | Methylsulfanyl(methyl- | — | 0.47 |
| | | sulfinyl)methane | | |
| | | Total: | 33.29 | 19.32 |

Other

| | | | | |
|---|---|---|---|---|
| 59 | 6921-35-3 | 3,3-dimethyl ethoxy | 0.75 | — |
| 60 | 109-67-1 | 1-pentene | 0.05 | — |
| 61 | 106-44-5 | P-cresol | 0.33 | 1.11 |
| 62 | 5418-86-0 | Tris(methylthio)methane | 0.10 | — |
| 63 | 120-72-9 | Indole | 3.11 | 4.23 |
| 64 | 96-76-4 | 2,4-di-tert-butylphenol | 0.19 | — |
| 65 | 108-95-2 | Phenol | — | 1.48 |
| 66 | 645-59-0 | Hydrocinnamonitrile | 0.09 | 0.14 |
| 67 | 100-52-7 | Benzaldehyde | 0.06 | — |
| 68 | 104-87-0 | P-methyl benzaldehyde | 0.88 | — |
| 69 | 19549-87-2 | 2,4-dimethyl-1-heptene | — | 0.47 |
| 70 | 1014-60-4 | 1,3-di-tert-butylbenzene | — | 0.69 |
| 71 | 1618-26-4 | Bis(methylthio)methane | — | 0.71 |
| 72 | 2432-83-9 | S-methyl octane thioate | — | 1.04 |
| 73 | 18252-44-3 | Beta-copaene | — | 0.32 |
| | | Total: | 5.57 | 10.19 |

Determination results show that 51 kinds of volatile substances are detected in the fermented pickles inoculated with Loigolactobacillus coryniformis subsp. coryniformis 523L5, mainly alcohols, acids and sulfur compounds. 36 kinds of volatile substances are detected in the naturally fermented pickles, mainly esters, sulfur compounds and alcohols. Compared with natural fermentation, for the fermented pickles inoculated with the Loigolactobacillus coryniformis subsp. coryniformis 523L5, the relative contents of alcohols are decreased, but the contents of butanol and n-hexanol are relatively high. Moreover, the variety of alcohols is richer, which can endow the pickles with the flavor such as wine fragrance, flower fragrance and rose fragrance. The pickles subjected to inoculation fermentation have the higher acid content than the naturally fermented pickles, more abundant acid types such as n-pentanoic acid, caprylic acid and heptylic acid, and the main acid substances are also changed from n-hexanoic acid to butyric acid, which provides more complex sour taste for the pickles and reduces the bad smell of fat in the pickles. The sulfur compounds play an important role in the flavor of fermented vegetables, and are also the main source of the flavor of the pickles. Inoculation fermentation can increase the content of sulfur compounds, especially dimethyl disulfide, which can provide fresh onion flavor and vegetable flavor for the pickles. The contents of the esters are decreased after inoculation, but new esters such as hexanoate and octanoate also appear. It is possible that after inoculation and fermentation, the species and quantity of the flora in the pickle solution change, resulting in the inability of some ester-producing bacteria to grow, and most volatile substances remain on precursors such as alcohols and acids.

### Example 6: Application of Loigolactobacillus coryniformis Subsp. Coryniformis 523L5 in Fermentation of Chinese Cabbages

1. Making of pickles: firstly, fresh Chinese cabbages were washed and drained, cut into small pieces of 7-8 cm in length, boiled and blanched for 1 min, and then taken out. 3% of brine by mass fraction was added according to 1 g: 2 mL, and the strain 523L5 wet bacterial cell suspension (wet bacterial cell concentration of 1 g/L) prepared by the method of Example 4 was added according to the mass of Chinese cabbages to the volume of the bacterial suspension of 100 g: 2 mL. Covering was made with plastic wrap, and sealing was performed. Natural fermentation without inoculation was used as a blank control group. Natural fermentation was carried out at a room temperature for 3 months.

2. Determination of pH and acidity of pickles: pH and acidity were tested by the method of Example 5.

TABLE 8
Determination results of pH and acidity of pickles

| | pH | | | Acidity | | |
|---|---|---|---|---|---|---|
| | 1 | 2 | 3 | 1 | 2 | 3 |
| Group | month | months | months | month | months | months |
| Natural | 4.16 | 3.91 | 4.36 | 2.02 | 2.17 | 1.47 |
| fermentation | | | | | | |
| Inoculation | 4.88 | 6.08 | 6.21 | 1.47 | 1.01 | 0.84 |
| fermentation | | | | | | |

The pH value of the pickles inoculated with the Loigolactobacillus coryniformis subsp. coryniformis 523L5 after completion of fermentation is much higher than that of the pickles fermented naturally, while the total acid content is just the opposite. Moreover, the pH value and total acid of the pickles inoculated with the Loigolactobacillus coryniformis subsp. coryniformis 523L5 in the first month of fermentation are similar to those in the third month of natural fermentation, but in the second month of fermentation, the pH value and the total acid are similar to those of excellent “Qianli Piaoxiang” pickles. Therefore, inoculation of the Loigolactobacillus coryniformis subsp. coryniformis 523L5 can accelerate the fermentation process of the pickles to a great extent and shorten the fermentation period.

What is claimed is:

1. The strain Loigolactobacillus coryniformis subsp. coryniformis 523L5, deposited in China General Microbiological Culture Collection Center on Jun. 24, 2024, accession number CGMCC No. 31070.

2. A method of using the Loigolactobacillus coryniformis subsp. coryniformis 523L5 according to claim 1 in improvement on quality of fermented pickles, comprising a step of fermenting raw materials of fermented pickles to obtain pickles.

3. The method according to claim 2, wherein raw materials of the fermented pickles comprise mustard stems and Chinese cabbages.

4. The method according to claim 2, comprising steps of:
washing and draining fresh raw materials of fermented pickles,
cutting the raw materials into pieces of appropriate sizes,
placing the pieces into a pickle jar,
adding brine with a mass concentration of 2-4%,
performing mixing,
inoculating the Loigolactobacillus coryniformis subsp. coryniformis 523L5,
performing sealing, and,
performing fermentation at 25-35° C. for 3 months.

5. The method according to claim 4, wherein the volume of the brine added is 1-2 mL/g based on the mass of the drained pickle raw materials.

6. The method according to claim 4, wherein the Loigolactobacillus coryniformis subsp. coryniformis 523L5 is inoculated in the form of a sterile physiological saline bacterial suspension of wet bacterial cells, wherein the wet bacterial cells have a concentration of 0.5-5 g/L, and an inoculation amount is 1-3 mL/100 g based on the weight of the drained pickle raw materials.

7. The method according to claim 6, wherein the wet bacterial cells are prepared by:
inoculating the Loigolactobacillus coryniformis subsp. coryniformis 523L5 into a MRS solid medium plate,
performing activation and culture at 37° C. for 48 h,
inoculating a cultured bacterial solution into a MRS liquid medium for culture at 37° C. for 16 h,
inoculating the cultured bacterial solution into a MRS liquid medium at an inoculation amount of 2% by volume concentration,
culturing in a shaker at 37° C. until OD_{600 nm} is 1.0,
centrifuging the culture solution at 4° C. and 5000 rpm for 10 min,
removing supernatant,
washing bacterial cells repeatedly with sterile physiological saline three times,
collecting wet bacterial cells, and
suspending the wet bacterial cells in sterile physiological saline of the same volume.
